(19) United States
(12) Patent Application Publication
(10) Pub. No.: US 2005/0019260 A1
(43) Pub. Date: Jan. 27, 2005

(54) Animal model for allergy

(76) Inventors: Elza Nicole Theresia Meeusen, Victoria (AU); Robert Juergen Bischof, Victoria (AU); Kenneth John Snibson, Victoria (AU)

Correspondence Address: CROWELL & MORING LLP;INTELLECTUAL PROPERTY GROUP, P.O. BOX 14300, WASHINGTON, DC, 20044-4300, US

(21) Appl. No.: 10/479,600

(22) PCT Filed: Jun. 4, 2002
(86) PCT No.: PCT/AU02/00715

(30) Foreign Application Priority Data
Jun. 4, 2001 (AU) PR 5444

(51) Int. Cl. A61K 49/00

(52) U.S. Cl. 424/9.2

(57) ABSTRACT

The invention relates to model systems for allergic conditions, and in particular to in vivo model systems in a large animal. The model systems of the invention are especially useful for providing large numbers of activated or non-activated eosinophils, for the discovery and evaluation of novel anti-inflammatory drug targets and for providing a model for the in vivo study of asthma and the effects of allergy treatments. In a preferred embodiment the animal is a sheep. In one embodiment, repeated infusion of house dust mite allergen (HDM) into the mammary gland is used to induce a specific allergic response, which is characterised by the recruitment of inflammatory cells, particularly eosinophils, into the mammary lumen; these cells can be harvested from peripheral blood and mammary lavage (MAL). In a second embodiment, the mammal is immunised with soluble antigen, for example by repeated subcutaneous immunisation, and then subjected to a single challenge with the same antigen administered directly to the lung.

This invention relates to model systems for allergic conditions, and in particular to in vivo model systems in a large animal. The model systems of the invention are especially useful for providing large numbers of activated or non-activated eosinophils, for the discovery and evaluation of novel anti-inflammatory drug targets and for providing a model for the in vivo study of asthma and the effects of allergy treatments. In a preferred embodiment the animal is a sheep.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

The prevalence of allergic diseases, in particular asthma, has increased dramatically in the last 20 years, doubling in Westernised societies. The severity of asthma is a particularly serious health issue in Australia, as it has one of the highest incidences of asthma in the world, with 1 in 4 children suffering from this condition.

Allergic asthma is an immunological disease associated with significant physiological changes in the lungs. The underlying immunological mechanisms directing the asthmatic response in the lungs are not clearly understood; however, a significant correlation between mast cells and eosinophils and the pathology of asthma has now been recognised. In particular, the pathophysiology of human asthma, including the development of airway hyperresponsiveness, is associated with the appearance of “activated” eosinophils and molecules released by these cells in bronchoalveolar lavage (BAL) fluid and in lung tissue (Walker et al, 1991; Desreumaux and Capron, 1996). Therefore there is a need in the art to investigate the processes involved in activation of eosinophils in an allergic response to a well-defined allergen, and to identify agents which can modulate this response.

Eosinophils are produced in the bone marrow and released into circulation where they migrate to inflammatory or parasite-infected sites. Stimuli present within the tissue microenvironment can cause eosinophils to become “primed” or “activated”, a state in which the ability of the eosinophil to carry out its effector functions is fully developed (Jones, 1993). One manifestation of eosinophil activation is an enhanced capacity to mediate antibody-dependent killing of helminth larvae. Increased respiratory burst activity, resulting in the release of toxic oxygen metabolites, and increased release of lipid mediators, such as leukotriene C4 and platelet activating factor, are associated with eosinophil activation and parasite killing. A classic marker for the activation of eosinophils is the release of pre-formed granule proteins, both spontaneously and in response to exogenous stimuli (Butterworth and Thorne, 1993). These granule proteins are known to be toxic to helminths.

In commonly used experimental systems in mice or humans it is very difficult to obtain large numbers of inflammatory cells, in particular eosinophils, because even in tissues where these cells are most prevalent they constitute only a small percentage of resident cells, and they can be isolated only with difficulty from these tissues. It is therefore not feasible to use normal eosinophils from these species for high through-put screening. Recently, an eosinophil cell line has been developed which could be used for screening, but since this is an immortalised cell line, it may react quite differently from normal cells, and does not provide an adequate model.

Animal models of disease allow defined and controlled investigations of key issues in disease progression to be carried out, with the possibility of being able to relate findings to the human situation. Studies in mice in particular have used powerful tools such as genetic knock-outs, knock-ins, and neutralisation of specific molecules to demonstrate an important role for the cytokines interleukin-4 (IL-4) and interleukin-5 (IL-5) [Grunig et al, 1998], and more recently interleukin-13 (IL-13) [Grunig et al, 1998; Wills-Karp et al, 1998], in the pathophysiology of asthma.

Unfortunately the smaller animal models, particularly those in mice, are limited, because they are not amenable to repeated sampling of cells, and/or because they yield only small numbers of cells for further studies. In addition, the development and physiology of the mouse lung is very different from that of human lung, and many of the pathological phenomena typical of human asthma are not adequately reproduced in the mouse models (Bice et al, 2000). Factors which may be responsible for the shortcomings of the mouse as a model for human asthmatic disease include poor development of smooth muscle structure associated with the lung airways, and poor responses to histamine in mice [Karol, 1994].

Sheep and other ruminants such as goats, and some non-ruminant animals such as pigs, have closer developmental and physiological similarities with humans than do mice, and are widely used as models for human physiological processes, including use of these animals in studies of immunological function. See for example “Handbook of Vertebrate Immunology” ed. P-P Pastoret et. al.,1998. In addition, large amounts of tissues and cells can be repeatedly harvested from a single such animal. It has previously been demonstrated that the allergic response in sheep lungs closely reproduces the development of the human asthmatic response, including a characteristic early- and late-phase asthmatic response, and bronchial hyperresponsiveness [Abraham et al, 1983; Fujimoto et al, 1996]. While sheep are now widely used to study the pharmacological effects of new anti-allergic compounds [Fujimoto et al, 1996; Fath et al, 1998; Abraham et al, 2000], so far none of the physiological studies in sheep have been combined with a detailed analysis of the associated immunological events. Although there have been reports of a model for allergic asthma using rhesus monkeys sensitised with house dust mite allergens (Schelegle et al, 2001) and dogs sensitised with Ascaris or ragweed allergens (Bice et al, 2000), there is still a need in the art for an IgE-specific large animal model of asthma. In particular, the monkey model requires repeated intranasal challenge following initial subcutaneous sensitisation, full anaesthesia of animals for measuring airway responsiveness, and is too expensive for large scale and detailed drug evaluation.

All of the previously-available sheep models of asthma have utilised acute allergic responses against an allergen derived from a nematode parasite, Ascaris suum, which is not an antigen relevant to asthma in humans. The use of Ascaris suum as the allergen in sheep asthma models was described about 20 years ago; no other allergens have been investigated in such a system, and no detailed immunological studies of the inflammatory response induced by the Ascaris antigen have been reported.

Ascaris-sensitised sheep are an inefficient physiological model for asthma, as only a small proportion of the sensitised sheep respond with the desired late-phase asthmatic response, which must be measured using complicated lung-function test equipment, and responders must be identified by trial and error. Different breeds of sheep may also react differently to Ascaris sensitisation; for example, only a small proportion of Australian merino sheep seem to respond. The expectation in the art was that sheep would only react to very strongly allergenic allergens such as Ascaris, and that therefore this approach is very strictly limited in its applicability to human allergies.

A sheep mammary infusion model has been described previously for the collection of large numbers of eosinophils for parasite killing assays (Rainbird et al, 1998; Duffus and Franks, 1980) and for the study of the cellular kinetics of an allergic-type response (Greenhalgh et al, 1996; Bischof and Meeusen, 2002). In these studies, parasite larvae or parasite extracts were infused through the teat canal into the mammary gland, and leukocytes thus induced to migrate into the mammary lumen were collected by infusion of sterile saline, followed by “milking” of the glands. While the basic technique has been known for some time, this method was mainly used for performing parasite killing assays, and more recently for basic studies of inflammation (Greenhalgh et al, 1996; Rainbird et al, 1998; Bischof and Meeusen, 2002). Its use for identifying novel target molecules or for high through-put in vitro screening assays has not previously been suggested, and is not a logical extension from the prior art.

It is now realised that long-term structural and functional changes to lung tissues, usually referred to as airway remodelling, in patients suffering from chronic asthma lead to significant increases in morbidity. The underlying biological processes involved in airway remodelling are poorly understood, and scientific progress in this area has been severely restricted by the lack of a suitable experimental system. Various mouse models of asthma exhibit some, but not all, of the morphological and functional lesions of the chronic human disease. A recently-described mouse model involving inhalation of ovalbumin aerosols shows subepithelial fibrosis, mucous cell hyperplasia, chronic inflammation of the lamina propria, and accumulation of intraepithelial eosinophils, but does not exhibit mast cell recruitment into the airway wall, or increase in smooth muscle mass (Kumar and Foster, 2001). Clearly, better animal models reflecting the human situation are required.

We have developed two novel approaches for the study of allergic responses in sheep, other ruminants, and pigs, which have distinct advantages over existing models for the discovery of novel therapeutic molecules and processes:
(a) a mammary infusion model for the collection of large numbers of eosinophils at different stages of activation, and
(b) an asthma model based on sensitisation with allergens which affect humans, such as an extract of the house dust mite, Dermatophagoides pteronyssinus (HDM),ragweed pollen, or food allergens.

### SUMMARY OF THE INVENTION

The invention generally provides an in vivo model system for an allergic condition, comprising a mammal of the order Artiodactyla, a non-human primate, or a member of the family Canidae, which has been subjected to allergic sensitisation with an antigen, with the proviso that the antigen is not one derived from Ascaris suum.

In a first aspect, the invention provides an in vivo model system for an allergic condition, comprising a mammal which has been subjected to sensitisation with an antigen or administration of a molecule involved in response to allergen, in which
a) the mammal is a female, and is sensitised by repeated administration of the antigen into the mammary gland; or
b) the mammal is of either sex, and is sensitised by administration of the antigen, followed by administration directly to the lung; or
c) the mammal is of either sex, and blood and tissue eosinophilia is induced by administration of a molecule involved in response to allergen,
in which the mammal is not a rodent, and the antigen is not one derived from Ascaris suum.

The antigen may be any antigen which is capable of inducing allergic sensitisation. Allergens contemplated to be suitable for use in the invention include those from house dust mite, animal danders such as cat, dog or bird dander, feathers, cockroach, grass pollens such as those from ryegrass or alternaria, tree pollens such as those from birch or cedar, other plant allergens, moulds, and household or industrial chemicals. Preferably the antigen is one which is associated with asthma in humans. In a particularly preferred embodiment the antigen is an extract of the house dust mite, Dermatophagoides pteronyssinus (HDM).

The order Artiodactyla includes sheep, goats, cattle, pigs, deer and antelope. Preferably the animal of this order is a ruminant, such as a sheep, goat, or cow, or is a pig. More preferably the mammal is a sheep or a goat.

The order Primates includes apes, Old World and New World monkeys, lemurs and tarsiers. Preferably the non-human primate is an ape or a monkey, more preferably a rhesus monkey (Macaca mulatta).

The family Canidae includes dogs, wolves, jackals, and the like. Preferably the animal of this family is a dog.

In one embodiment of this method, repeated infusion of house dust mite allergen (HDM) into the mammary gland is used to induce a specific allergic response, which is characterised by the recruitment of inflammatory cells, particularly eosinophils, into the mammary lumen; these cells can be harvested from peripheral blood and mammary lavage (MAL). The development of eosinophilia in blood and tissues after allergen challenge is due to the induction of host regulatory molecules (e.g. cytokines) which drive the increased production of eosinophils from the bone marrow and their recruitment via the blood to the allergen-challenged tissue. Mammary and/or peripheral blood eosinophilia can therefore also be induced directly by administering host molecules involved in the response to allergens(e.g. cytokines such as interleukin-5 and eotaxin) (Foster et al, 2001).

The large numbers of inflammatory blood and MAL cells collected by these procedures can be used for the following applications:
(a) Identification of processes and molecules differentially active or expressed in “activated” and “non-activated” eosinophils and other inflammatory cells;
(b) Identification of processes and molecules involved in the recruitment of eosinophils and other inflammatory cells;
(c) Identification of processes and molecules involved in degranulation of eosinophils and other inflammatory cells;
(d) In vivo screening and testing of new anti-inflammatory drugs and therapies; and
(e) Use of inflammatory blood and MAL cells, including but not limited to eosinophils, for in vitro screening assays for the development of new anti-inflammatory or anti-degranulation/activation drugs.

In a second embodiment, the mammal is immunised with soluble antigen, for example by repeated subcutaneous immunisation, and then subjected to a single challenge with the same antigen administered directly to the lung. Preferably the lung challenge is administered using a fibre-optic bronchoscope; this permits localised delivery of the antigen challenge deep into the caudal lobe of the lung. For repeated sensitisation and evaluation of airway mechanics, the antigen is preferably administered as an aerosol.

This embodiment of the model of the invention provides a direct model system for the study of asthma, in which broncho-constriction can be measured in un-anaesthesised animals. The effects of chronic allergen exposure, including tissue remodelling, can be examined. Airway remodelling is also characteristic of chronic asthma. This model is also suitable for in vivo testing of the efficacy of candidate drugs or drug delivery methods for the treatment of asthma, including the testing of long-term therapeutic procedures. This model is also suitable for studies of airway remodelling.

The model of the invention provides a convenient system in which a reproducible inflammatory response can be induced, and can be studied with significantly greater ease than has hitherto been possible.

The present application describes for the first time:
(a) the use of a major human allergen, house dust mite extract (HDM), in a sheep asthma model,
(b) a correlation between high IgE responder (atopic) sheep and the induction of a sustained allergic response (eosinophil recruitment) in the lung after challenge, consistent with the human situation, and
(c) the chronic stimulation of sheep lungs with HDM to induce tissue remodelling changes of the kind which are typical of chronic asthma in human patients.

As a result of the well-known physiological similarity between sheep and human respiratory systems, and between humans and other primates, we expect that the sheep models can readily be extended to non-human primates. Similarly, dogs have widely used in studies of allergy and asthma; see for example Bice, et al. (2000). We therefore also expect that the sheep models can be extended to dogs.

The allergens used in the model according to the invention may be administered by any suitable route, and the person skilled in the art will readily be able to determine the most suitable route and dose for the condition to be induced For example, in the mammary infusion model antigen is infused directly into the teat canal. For the lung model, initial sensitization may be effected by a variety of routes; however, preferably the antigen is administered by oral, subcutaneous, intradermal or intramuscular injection, more preferably by subcutaneous injection with alum as adjuvant. Optionally other adjuvants or immunomodulators such as Freund's adjuvant, iscoms or cytokines may be used. Many alternative adjuvants are known in the art.

It is known that interleukin-5 (IL-5) induces eosinophilia and eotaxin recruits eosinophils into tissues; for example IL-5 gives a high eosinophil response in a variety of animal models (Foster et al, 2001). We therefore expect that the model of the invention can be reproduced by treatment of animals with IL-5 or eotaxin. Preferably this modification is used with the mammary model of the invention.

The nature of the carrier or diluent, and other excipients, which are used for the allergen will depend on the allergen and the route of administration, and again the person skilled in the art will readily be able to determine the most suitable formulation for each particular case. For example, methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

For the purposes of this specification it will be clearly understood that the word “comprising” means “including but not limited to”, and that the word “comprises” has a corresponding meaning.

### BRIEF DESCRIPTION OF THE FIGURES

FIG. 1 illustrates the eosinophilic responses observed following HDM infusions of the mammary gland (n=3):
(a) MAL cell suspensions;
(b) Peripheral blood (PBL).

FIG. 2 shows the changes in surface marker expression on MAL eosinophils with time after infusion, as assessed by flow cytometry. Values are mean±standard deviation (n=3). MFI=mean fluorescence intensity.
(A) VLA-4, (B) L-selectin, (C) LFA-1, (D) CD11b, and (E) CD44.

FIG. 3 illustrates the changes in MAL lymphocyte subpopulations after HDM infusions. Values are mean±standard deviation (n=3).

FIG. 4 shows the HDM-specific total serum immunoglobulin response to HDM infusions: the results shown are from samples taken prior to the commencement of HDM mammary infusions (open circles) and 7d after the third HDM infusion (closed squares).

FIG. 5 is a schematic representation of the sensitisation and lung challenge protocols used in the invention.

FIG. 6 shows the effect on specific Ig classes of allergic sensitisation of sheep to HDM. Results are shown for IgE (A), IgG1 (B) and IgG2 (C).

FIG. 7 shows responses to lung challenge with HDM following allergic sensitisation (n=3).

FIG. 8 shows peribronchiolar airway wall remodelling changes in house dust mite (HDM)-challenged compartments in responder sheep after chronic allergen challenge. The panels A-C show log/log plots of collagen content in bronchiole walls against lumen area. Trend lines were calculated in Microsoft Excel software, based on power regression. Bronchiole lumen size was measured by the area circumscribed by the bronchiolar basement membrane. Panels A and B show collagen data derived from an image analysis performed on responder sheep. Significant difference for HDM challenge (full circles) v untreated internal control (open circles) compartments: p<0.0005, and p<0.005 respectively. Panels C and D show collagen data from representative saline-challenged control sheep and nonresponder HDM-challenged sheep respectively. Saline challenge(full circles)v untreated internal control(open circles) compartments p NS., HDM challenge v untreated internal control compartments p NS. Panels E and F show corresponding log/log plots of peribronchiole connective tissue (E) and bronchiolar smooth muscle (F)of A & B responder sheep for HDM challenged (full circles) and untreated internal control (open circles) lung compartments.

FIG. 9 shows that chronic challenge with house dust mite (HDM) induces airway wall remodelling-like responses in sheep lungs. The panels depict histology of Masson's trichrome-stained sections of similar sized bronchioles from HDM challenged (right panel) and untreated control (left panel) lung compartments in the same sheep. a, alveoli; c, collagen (Masson's trichrome-stained); ce, columnar epithelium; e, cuboidal epithelium; g, goblet cell (additional staining shows that these are predominantly Alcian blue positive); l, lymphocyte; sm, smooth muscle. Magnification for both panels ×100 and insets ×400. In contrast to the control, note in the challenged bronchiole the presence of increased collagen and smooth muscle, increased numbers of goblet cells, and columnar rather than cuboidal epithelium (see high magnification inset of the boxed region).

FIG. 10 shows a high magnification view of the changes in bronchiolar epithelium following chronic challenge with HDM (left panel) compared to the unchallenged lung compartment (right panel) of the same sheep, illustrating the changes resulting from airway remodelling. Masson's Trichrome stain, magnification ×400.

FIG. 11 shows the results of Northern blot analysis of galectin-14 mRNA levels in isolated leukocytes and whole tissue. Total RNA from macrophage (M)-, neutrophil (N)-, or eosinophil (E)-rich MAL cell populations, or from lung tissue (L) or BAL cells (B) were used. The lung tissue and BAL cells were collected from sheep that had been sensitized with HDM and challenged 48 h earlier in the left lung lobe with HDM and in the right lung lobe with sterile PFS (Treated Sheep). Control sheep received sterile PFS only in both lung lobes (Controls). 18 S rRNA is shown to correct for loading errors. The results shown are representative of three treated and three control sheep.

FIG. 12 shows the results of SDS-PAGE and Western blot analysis of recombinant galectin-14 and endogenous proteins. Cleaved and purified recombinant galectin-14 (rGal-14) was analyzed using Coomassie Blue-stained SDS-PAGE (Panel A), or Western blot using a monoclonal antibody directed against galectin-14 (Panels B and C). MAL eosinophils (E), MAL neutrophils (N), lymph node (LN) lymphocytes (L), BAL macrophages (M) from control lungs; BAL cells containing 5% eosinophils after local HDM challenge (HDM). The far right panel (C) shows the presence of galectin-14 in cell-free MAL fluid of a sensitized sheep before (S) and after (SC) HDM challenge of the mammary gland. The arrow points to the position of monomeric galectin-14. All samples were run under reducing conditions.

FIG. 13 shows the resistance to pulmonary airflow increases after inhalation challenge with HDM. Airways resistance is expressed as a percentage of the baseline resistance value (18.6 cmH₂O 1⁻¹ s).

### DETAILED DESCRIPTION OF THE INVENTION

While the invention is specifically described herein with reference to two embodiments of the model in sheep, it will be appreciated that because of their close evolutionary relationship, the biological responses of sheep are very similar to those of other members of the order Artiodactyla. In particular, sheep and goats react in very similar ways.

Divergence of mammalian proteins is highest amongst the ligands and receptors of immunological molecules such as cytokines, cytokine receptors and leukocyte surface antigens. Homologous molecules in different species have a common ancestral gene and, depending on the evolutionary differences, are likely to have the same function and biochemical characteristics. Cross-reactivity between ruminants such as sheep, goats, and cattle is very extensive because of their close phylogenetic relationship (Naessens et al, 1997). Ruminants and other large animals, such as pigs and horses, also have closer evolutionary relationships with humans than do mice, and their immune proteins therefore share greater biological characteristic and sequence homologies with humans than do equivalent mouse molecules (Naessens et al, 1997; Villinger et al, 1995).

Similarly, although the embodiments specifically described herein utilise one specific allergen, it would be expected that other relevant human allergens could also be used in these systems, using the same methodology described for HDM but with the optimal antigen dose being determined by routine methods for each individual allergen.

The mammary infusion model system of the invention provides an in vivo model of inflammation for the study of allergic responses. The model allows non-invasive and repeated sampling of inflammatory cells following tissue migration into the lumen of the mammary gland, and offers many advantages for detailed examination of the in vivo recruitment of eosinophils during allergic-type responses [Greenhalgh et al, 1996; Bischof and Meeusen, 2002]. This model is particularly useful, because the washes from stimulated mammary glands provide a rich source of cells which have traversed both endothelial and epithelial barriers, and thus are similar to cells found in the bronchial lumen during pulmonary diseases such as asthma. Populations of 2-5×10⁷ cells can routinely be obtained.

In addition, simultaneous collection of leukocytes from the peripheral blood of the same animal allows detailed analysis of the changes in surface phenotype of cells before and after tissue migration, with only minimal in vitro manipulation. Depending on the stimulus used, our experimental system provides a hitherto unavailable supply of cells which is highly enriched in vivo for eosinophils which are either activated or non-activated [Greenhalgh et al, 1996; Rainbird et al, 1998], and therefore offers an ideal system to study activation-induced changes in eosinophils. In addition to antigens, host molecules such as cytokines can also be administered both in vivo and in vitro to induce eosinophilia or eosinophil activation respectively. Moreover, a different stimulus such as lipopolysaccharide (LPS) may optionally be used to induce migration of large numbers (up to 10⁹) of almost pure neutrophils into the mammary lavage [Greenhalgh et al, 1996].

The asthma model of the invention provides a number of advantages over smaller animal models of asthma. For example, bronchoconstriction can be measured in un-anaesthesised animals, so that there are no confounding effects resulting from the use of anaesthetic agents. Using a fibre-optic bronchoscope, it is possible to take multiple samples and measurements from one or more lung compartments in one animal; such a technique cannot be used with small animals such as mice. This approach is important, as it allows each animal to serve as its own control, thus reducing the effect of inter-animal variability associated with an out-bred population. A further important advantage of large animals such as sheep, goats and cattle is their longevity in comparison to rodents, which enables assessment of the effects of chronic allergen exposure, including tissue remodelling, and long-term therapeutic procedures.

The invention will now be described in detail by way of reference only to the following non-limiting examples and drawings.

Materials and Methods

Animals

For both the mammary infusion model and the lung model, mature non-lactating merino ewes (2-3 years old and previously lactating) and 4-5 month old female merino-cross lambs were purchased from a commercial farm. All animals were treated with the anthelminthic Nilverm (Cooper's Animal Health, North Ryde, Australia) prior to the experiment to eliminate existing parasites. The sheep were housed in pens and fed commercial sheep pellets (Barastoc, Pakenham, Australia).

Preparation of House Dust Mite for Immunisation and Challenge

The ovine mammary infusion model and lung model described in detail herein are based on sensitisation to and challenge with house dust mite (HDM; Dermatophagoides pteronyssinus). Dried HDM (mites+faecal matter) was obtained from the Commonwealth Serum Laboratories (CSL) Ltd., VIC, Australia. A soluble solution of HDM was prepared by grinding HDM in 5 ml sterile pyrogen-free saline (PFS; Baxter Healthcare Pty. Ltd, NSW, Australia), followed by centrifugation at 14,000 rpm and removal of the supernatant (soluble solution) from particulate matter. Using a syringe, the HDM solution was sterile-filtered through a 0.2 μm filter (Gelman Sciences, MI, USA) and adjusted to working strength as described below with the addition of sterile PFS.

Determination of HDM-specific Serum Immunoglobulin Responses

For the determination of HDM-specific total immunoglobulin (Ig), IgG1 and IgG2, serum samples were assayed by enzyme-linked immunosorbent assay (ELISA) as follows. Wells of a 96-well microtitre plate (Nunc-Immuo Maxisorb, Nunc Intermed, Denmark) were coated with 50 □l of 50 μg/ml HDM antigen in coating buffer (150 mM Na₂CO₃, 350 mM NaHCO₃, 0.1% sodium azide (pH9.6)), and incubated in a humidified box overnight at room temperature (RT). Following 3 washes in wash buffer (0.05% Tween-20 in PBS), plates were blocked for 60 min at 37° C. with the addition of 200 μl blotto (2% w/v BSA in PBS) to each well. Plates were washed 3 times, 100 μl serum (diluted 1/100 in blotto) added to each well and plates were incubated for 90 min at 37° C. From this point the plates were handled separately for the detection of either total Ig, IgG1 or IgG2.

For detection of HDM-specific total Ig, plates were again washed prior to the addition (50 μl/well) of horseradish peroxidase (HRP)-conjugated anti-sheep Ig (Dako, CA, USA; 1:2000 in blotto). After incubation for 60 min at 37° C., plates were washed and developed with the addition of 100 μl/well 1 mg 3′, 3′, 5′, 5′-tetramethyl-benzidine dihydrochloride hydrate substrate (TMB, Sigma) dissolved in 1 ml 100 mM citric acid, 2 ml 500 mM acetate buffer, 5 μl H₂O₂ and 7 ml MQ-H₂O. After 10 min the reaction was halted by the addition of 50 μl/well of H₂SO₄. Isotype-specific ELISA was performed for detection of serum IgG1 and IgG2. Following incubation with serum as described above, plates were washed followed by incubation with 50 μl/well of undiluted anti-IgG1 or anti-IgG2 monoclonal antibody (mAb) culture supernatants (gifts from K. Beh, CSIRO, VIC., Australia) for 60 min at 37° C. Plates were again washed and incubated with HRP conjugated rabbit anti-mouse Ig (Dako; 1:2000) for 60 min at 37° C., then washed and developed as described above. For each of the ELISAs performed, optical density (O.D.) was determined with a TitreTek Multiscan MCC plate reader using a dual wavelength (A₄₅₀-A₆₉₀).

HDM-specific IgE serum responses were assessed by ELISA. HDM antigen-coated plates, prepared as described above, were washed 6 times with 150 mM NaCl, 0.05% Tween 20 in 10 mM phosphate buffer, pH 7.2 (PBST), then blocked with 250 μl blotto for 60 min at RT. Equal volumes of serum and 80% NH₄SO₄ (BDH) solution, prepared from a saturated solution of NH₄SO₄ in distilled water, were mixed for 10 sec using a vortex mixer. The sample was vortexed again at 15 min, then centrifuged at 30 min in a microcentrifuge (13,000 rpm for 10 min). NH₄SO₄-treated serum samples were diluted 1/20 in 0.05% Tween 20/distilled water; 100 μl of diluted sample was added to the coated plates in triplicate and plates incubated overnight at RT. Plates were again washed 6 times, followed by incubation with 50 μl/well of anti-IgE mAb culture supernatants (clone XB6/YD3, undiluted; Agresearch, NZ) for 4 h at RT. Plates were again washed and incubated with HRP-conjugated rabbit anti-mouse Ig (gamma chain specific, Sigma; 1:1000) for 60 min at 37° C., then washed and developed as detailed above. The reaction was halted after 30 min by the addition of 50 μl of H₂SO₄/well, and plates were read as described above.

Flow Cytometry

Monoclonal antibodies (mAbs) against the sheep cell surface molecules CD1, CD2, CD4, CD5, CD8, CD45R, WC1, WC2, CD45, CD25, MHC class II, LFA-1, CD11b, CD44, VLA-4, L-selectin, β1- and β7-integrin were used (Naessens, et al, 1997) The mAb SBU-3 (Lee et al., 1985) does not react with sheep leukocytes, and was used as a negative control.

MAL and peripheral blood leukocytes were counted using a Coulter counter® (Coulter Electronics, Luton, UK) and resuspended to 2-3×10⁷ cells/ml in wash buffer (1% BSA/0.05% azide/PBS) on ice. Cells were preincubated with 5% normal sheep serum and 5% foetal calf serum (CSL) for 10 min (on ice), then transferred in 50 μl aliquots to a 96-well V-bottomed plate. To each well, 50 μl of mAb (undiluted culture supernatant) was added, and cells were incubated for 30 min, then centrifuged and washed three times with wash buffer prior to incubation with fluorescein (FITC)-conjugated sheep anti-mouse F(ab′)₂ Ig (Silenus, Vic., Australia; 1:80 in wash buffer). All staining incubations were performed at 4° C. on a Dynatech microshaker (Selbys, Melbourne, Australia). After further washes, cells were preincubated with 5% normal mouse serum (Chemicon, CA, USA) in wash buffer for 10 min prior to the secondary staining using a biotinylated anti-CD4 mAb (Balic et al, 2000), followed by three washes and incubation with streptavidin-phycoerythrin (PE)-conjugate (Biosource Int, Camarillo, USA; 1:800 in wash buffer). Cells were then washed as before, fixed in 3% formaldehyde in PBS and analysed on a FACSCalibur® instrument (Becton-Dickinson, Mountain View, USA) using Cellquest® software (Becton-Dickinson).

### EXAMPLE 1

Sheep Mammary Infusion Model

Sheep were primed by 3-4 infusions of the mammary glands at 2-week intervals with 5 ml of a soluble preparation of HDM (0.2 mg/ml in sterile PFS), then rested for 3-4 weeks prior to the experimental challenge. Mammary infusions were performed using a 10 ml syringe fitted with a blunted 22-gauge needle. The tip of the needle was gently rotated into the teat canal, followed by infusion of the HDM preparation. At 24 h and 96 h post-HDM challenge, MAL cell suspensions (2-5×10⁷ cells) were gently “milked” from the mammary glands after the infusion of 8 ml sterile PFS. On ice, MAL cells were washed and centrifuged (400 g, 5 min) twice with 1% bovine serum albumin (BSA, fraction V; Trace Biosciences, VIC, Australia) in phosphate-buffered saline (PBS) prior to immunostaining as described below.

Immediately preceding the collection of MAL cells, 20 ml blood was drawn from the jugular vein of sheep into a plastic tube containing ethylenediamine tetraacetic acid (EDTA; BDH Merck, VIC, Australia). Red blood cells were lysed with the addition of Tris-buffered ammonium chloride (TAC; 170 mM Tris, 160 mM NH₄Cl, pH 7.2) at 39° C., and leukocytes washed twice with PBS, resuspended in 1% BSA/PBS and stored on ice prior to immunostaining. Cytospots of MAL cells and blood smears were prepared and stained with Wright's stain (Sigma, Castle Hill, Australia) for differential leukocyte cell counts. Additional blood samples were collected prior to the first and 7d following the third mammary infusion of HDM, and allowed to clot at 37° C. for 60 min.

Serum samples were centrifuged and stored frozen at −20° C. for later analyses of serum immunoglobulin (Ig) responses by ELISA.

### EXAMPLE 2

Allergic-type Responses to HDM in the Mammary Gland

Sheep were primed by three HDM infusions of the mammary glands at 2-week intervals. MAL cell suspensions were gently milked from the glands at 24 h and 96 h following each HDM infusion, and cytospots were prepared and stained with Wright's stain for the enumeration of eosinophils.

Peripheral blood (PBL) was collected prior to infusion; eosinophils were enumerated using a Coulter counter, and blood smears were prepared and stained with Wright's stain. HDM infusions into the mammary gland induced a rapid recruitment of eosinophils into the MAL, increasing from 5-40% of cells after the first infusion to 75-90% after 3-4 infusions, as shown in FIG. 1A. The percentage of eosinophils recovered in the MAL was comparable at the 24 h and 96 h time points over the priming period. The rapid and progressive recruitment of eosinophils into the MAL was accompanied by elevated blood eosinophils, as shown in FIG. 1B.

The expression of cell surface antigens on eosinophils and lymphocytes obtained from MAL following HDM infusions was analysed by flow cytometry. Eosinophils were gated out on FSC and SSC characteristics and analysed for percentage (%) positive and mean fluorescence intensity (MFI) of adhesion molecule expression. At 24 h post-HDM infusion, most MAL eosinophils (>85%) expressed the cell surface molecules VLA-4, L-selectin, LFA-1, CD11b and CD44, as illustrated in FIGS. 2A-E. At 96 h post-HDM infusion there was a significant reduction in the percentage of MAL eosinophils expressing VLA-4 (FIG. 2A), L-selectin (FIG. 2B) and CD11b (FIG. 2D). The intensity of VLA-4 expression on MAL eosinophils was significantly increased at 96 h compared to 24 h post-HDM infusion (FIG. 2A). These changes were observed after both the primary and repeated infusions.

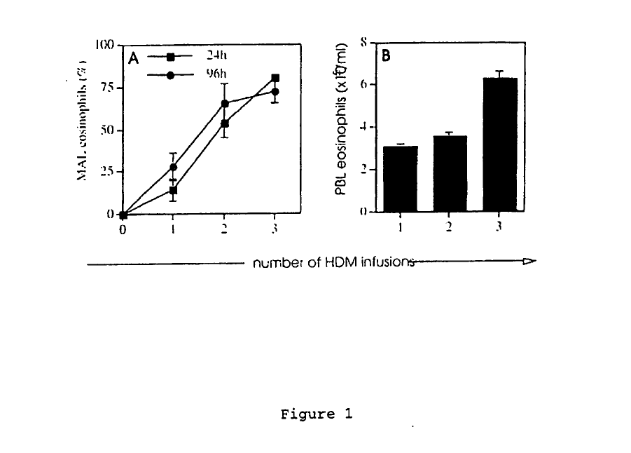

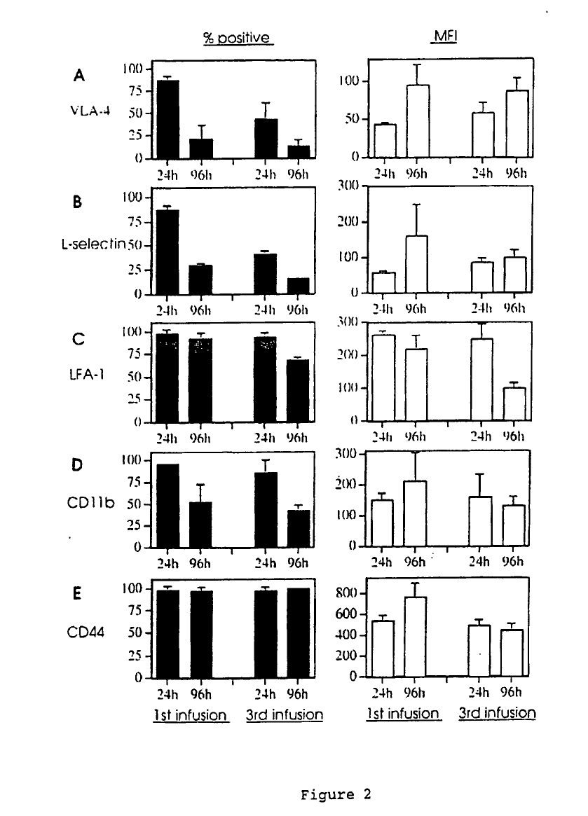

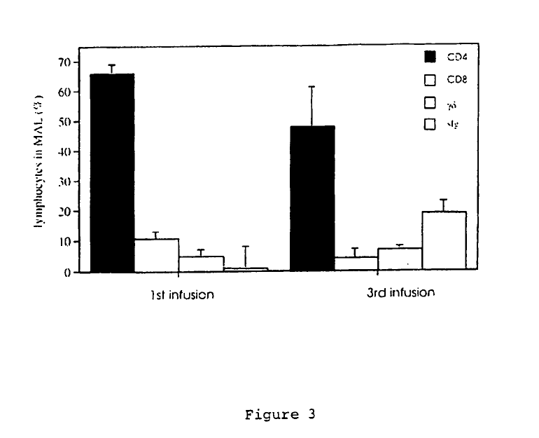

Lymphocytes were gated out on FSC and SSC characteristics and stained with mAbs against CD4⁺, CD8⁺, γδ-TCR⁺ and sIg⁺. Flow cytometry analysis of the lymphocyte subpopulations in the mammary gland lumen after HDM infusion indicated that CD4⁺ T cells were the predominant MAL lymphocytes. As shown in FIG. 3, most of these lymphocytes were in an activated state, as indicated by cell surface expression of CD25 (IL-2Rα) and MHC class II molecules. It was also noted that the proportion of B lymphocytes (sIg⁺) increased significantly in the MAL after priming (p<0.05).

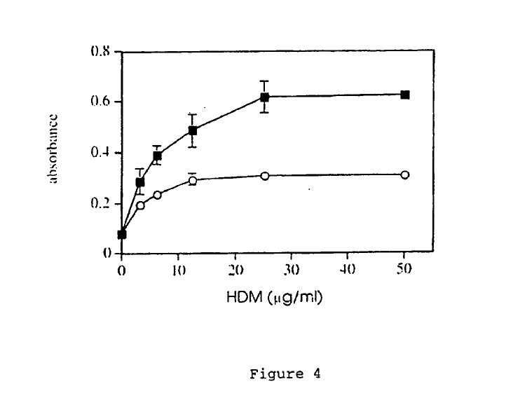

Serum from peripheral blood collected prior to the commencement of HDM mammary infusions (open circles) and 7d after the third HDM infusion (closed squares) was used for determination of HDM-specific total Ig responses by ELISA. Repeated HDM infusions had a systemic effect, and HDM-specific Ig was detected in serum collected from ewes after HDM infusions, as demonstrated in FIG. 4, which shows the HDM-specific total serum immunoglobulin response to HDM infusions.

### EXAMPLE 3

Sheep Lung Allergic Sensitisation Model

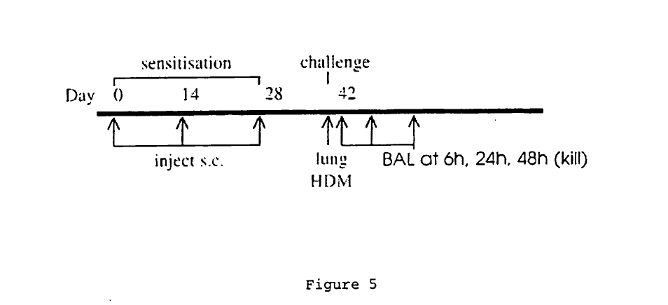

A schematic representation of the general sensitisation and lung challenge protocol is shown in FIG. 5. Groups of 5 sheep were immunised with a soluble preparation of HDM (0, 5, 50 or 500 μg in saline/Alum; 1:1); 3×subcutaneous (s.c.) injections made into the upper foreleg at 2 week intervals. Sheep were then rested for 2 weeks prior to a single lung challenge with HDM on Day 42 of the experiment. Serum samples were collected prior to each injection and at 7d and 14d after the last injection for assessment of HDM-specific serum antibody responses. During the experimental lung challenge procedure, unsedated sheep were restrained in a custom-made body sheath and head harness, and tethered in a modified metabolism cage.

Allergen challenge was administered directly to the lungs using a fibre-optic bronchoscope (Pentax FG-16X) for localised delivery of a soluble preparation of HDM (1 mg in 5 ml PFS at 39° C.) deep into the left caudal lobe of the lungs. The HDM preparation was delivered into the lung via the biopsy port of the bronchoscope using a 10 ml syringe.

One week prior to the experimental lung challenge, baseline BAL samples were collected via the bronchoscope from all sheep, by slow infusion and withdrawal of 5×10 ml aliquots of PFS (39° C.). Sequential BAL samples, typically returning 1-20×10⁶ cells, were collected from the left lungs at 20 min, 6 h, 24 h and 48 h post-challenge, by gentle instillation and withdrawal of 10 ml of PFS (39° C.).

Sheep were killed at 48 h post-challenge with an intravenous injection of 20 ml lethabarb (pentobarbitone sodium, 325 mg/ml; Virbac, VIC, Australia). Lung biopsy samples, collected using the biopsy port of the bronchoscope, and peripheral blood samples were also collected at these time-points. On ice, BAL cells were washed and centrifuged (400 g, 5 min) twice with 1% BSA/PBS prior to immunostaining. Cytospots of BAL cells and blood smears were prepared and stained with Wright's stain for differential leukocyte cell counts.

### EXAMPLE 4

Responses to HDM in the Sheep Lung Model

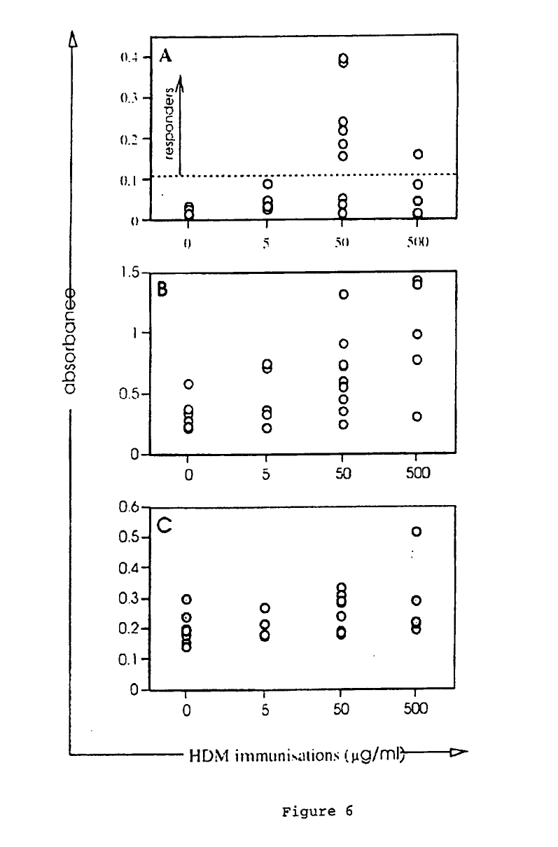

Groups of sheep were given 3×s.c. immunisations with HDM at different doses, and blood serum was collected for analysis of HDM-specific serum responses. Sheep were immunised s.c. (3× at 2 week intervals) with 1 ml of 0, 5, 50 or 500 μg HDM with alum as adjuvant, and HDM-specific IgE, IgG1 and IgG2 were assayed by ELISA in blood serum samples taken at 7d after the third HDM-specific immunisation. FIG. 6 shows the effect of allergic sensitisation of sheep to HDM on specific Ig classes.

IgE responses were strongest in the group immunised with 50 μg/ml HDM, as shown in FIG. 6A. In contrast, HDM-specific IgG1 responses were maximal when immunised at 500 μg/ml, as shown in FIG. 6B. No differences in IgG2 levels were detected, as shown in FIG. 6C.

On the basis of the results of this experiment, sheep were allocated into separate groups for assessment of their response to a challenge with HDM administered directly to the lungs. Sheep were divided into “responders” (immunised, IgE⁺; FIG. 6A), “non-responders” (immunised, IgE⁻, ie no IgE response) and “controls” (not immunised, IgE⁻). Groups of 3 sheep classed as “responders” were compared with “non-responders” and “controls” following lung challenge with HDM. Sheep were immunised s.c. (3× at 2 week intervals) with 1 ml of 0, 5, 50 or 500 μg HDM with alum as adjuvant, and HDM-specific IgE was assayed by ELISA in blood serum samples taken at 7d after the third immunisation. All the sheep were given a lung challenge with HDM delivered as a solution (1 mg in 5 ml PFS) via a bronchoscope deep into the left caudal lobe of the lungs. BAL was collected at 6 h, 24 h and 48 h post-challenge for the enumeration of BAL eosinophils.

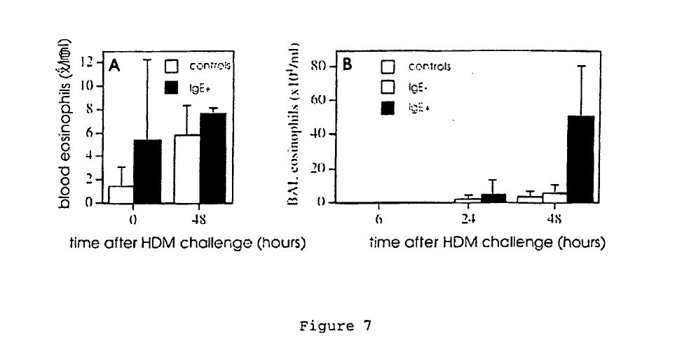

Data are presented in FIG. 7 as mean±s.d. (n=3 sheep/group). There was a trend toward increased peripheral blood eosinophil numbers before and after lung challenge in responder sheep (IgE⁺) compared to control sheep, as shown in FIG. 7A. Eosinophils appeared in the BAL at 24 h and 48 h following lung HDM challenge. In responders there was a dramatic influx of eosinophils into the BAL at 48 h compared with non-responders and controls, as shown in FIG. 7B.

### EXAMPLE 5

In Vitro Measurements of Eosinophil Activation and Degranulation for Drug Screening

Collection of Eosinophils From Blood or Mammary Glands.

Highly enriched preparations of eosinophils are obtained from the blood or mammary glands of allergen-sensitised sheep described in examples 1&2. Eosinophils may be further purified from these cell suspensions using standard cell purification techniques such as density gradient separation, flow cytometric cell sorting, and negative or positive selection with antibodic

It is well established in murine models that the host-derived cytokine, interleukin-5 (IL-5), is responsible for the marked increase in blood and tissue eosinophils (eosinophilia) induced by allergens and that the experimental administration IL-5, e.g. through injection of recombinant IL-5 or by overexpressing the IL-5 gene, can directly result in increased blood and tissue eosinophil numbers even in the absence of allergic stimulation (Foster et al. 2001). As a logical step, highly enriched blood and tissue eosinophils may therefore also be obtained in mammals (dogs, sheep, goat, cattle, pig, monkey) other than rodents, through injection of recombinant IL-5. In particular, sheep may be injected with recombinant IL-5 at concentrations from 0.5-10 μg/kg/day for 1-5 days by intravenous, subcutaneous or intramuscular routes. Peripheral blood enriched for eosinophils may be collected after IL-5 treatment and used in the in vitro assay either directly or after purification of eosinophils using standard procedures describes above. Eosinophils of IL-5 treated sheep may also be concentrated into the mammary gland by infusion of allergen into the gland as described in example 1, or by infusion of host derived chemotactic cytokines. In particular, the host cytokine eotaxin has been shown to be responsible for the specific recruitment blood eosinophils into tissues and bronchoalveolar lavage (Fos et al. 2001). HDM or 0.5-100 μg of recombinant or synthetic eotaxin may be infused into the mammary gland of sheep with high peripheral blood eosinophil levels. Mammary lavage (MAL) cells enriched for eosinophils may be harvested from these allergen chemokine treated glands according to procedures described in experiment 1. Eosinophil-enriched or purified cell preparations may then be used in an in vitro assay for drug screening, as detailed below.

Eosinophil Peroxidase (EPO) Release Assay

Peroxidase released by degranulating eosinophils is assayed according to a published procedure (Mengazzi, et al, 1992), with some modifications. Briefly, duplicate samples (50 μl) of eosinophils (5×10⁴ cells) are placed in the wells of a 96 well microtitre plate.

Calcium ionophore A23187 (Sigma) is dissolved at 18 mM in dimethyl sulphoxide (DMSO) and stored in aliquots at −20° C. 2-acetyl-1-hexadecyl-sn-glycero-3-phosphocholine (PAF; Sigma) is dissolved at 3 mM in chloroform-methanol (9:1, v/v) and stored at −20° C. in a nitrogen atmosphere. Cytochalasin B, an inhibitor of eosinophil degranulation, is dissolved at 10 mg/ml in DMSO. Calcium ionophore A23187, PAF and cytochalasin B are used at 5 μM, 1 μM and 5 μg/ml, respectively.

After the addition to each well of 20 μl of control buffer with or without the appropriate stimulus, the plate is incubated at 37° C. for 30 min. Following incubation, the peroxidase reaction is started by adding 70 μl of 3 mM TMB, 8.5 mM potassium bromide in 50 mM sodium acetate buffer pH 5.4, and 60 μl of 0.3 mM hydrogen peroxide. After 3 min of incubation at RT the reaction is stopped by addition of 50 μl 2M H₂SO₄. Absorbance is read at 450 nm on a microplate reader.

The aliquot of peroxidase activity released into the extracellular environment is expressed as a percentage of the total peroxidase activity of 5×10⁴ eosinophils. The total peroxidase activity (100%) is extrapolated from the linear part of calibration curves prepared by assaying the peroxidase activity of different numbers of eosinophils in the presence of 0.01% Triton X-100.

Results may be expressed as a percentage of control extracellular peroxidase, or as % change in optical density. The effect of various potential drug inhibitors of degranulation may be measured in this system by adding a range of concentrations of the test drugs to the degranulation assays.

A number of other measures of eosinophil activation and mediator release established for other species, are known in the art, including measuring granule release by ELISA, measuring oxidative burst and measuring lipid mediator biosynthesis. These may readily be adapted to assays of sheep mammary lavage and blood eosinophils.

### EXAMPLE 6

Model for Airway Wall Remodelling in Chronic Asthmatics

Sheep were sensitized to HDM as outlined in Example 4. Repeated allergen challenges were administered to sheep which displayed high IgE responses to HDM. Three control or saline-challenged sheep, and four atopic (high HDM IgE responder) HDM-challenged sheep, were challenged twice weekly in the caudal lobe of the left lung over a 6 month period. The sheep were challenged with HDM at 200 μg/ml PFS delivered via the biopsy port of a bronchoscope, as outlined in Example 4. In individual sheep, the equivalent compartment in the right lung was used as an untreated internal control. Seven to 14 days after the last challenge, sheep were killed, and their lungs removed and subjected to inflation fixation to preserve airway architecture. A detailed morphometric computer-aided image analysis was performed on histological samples.

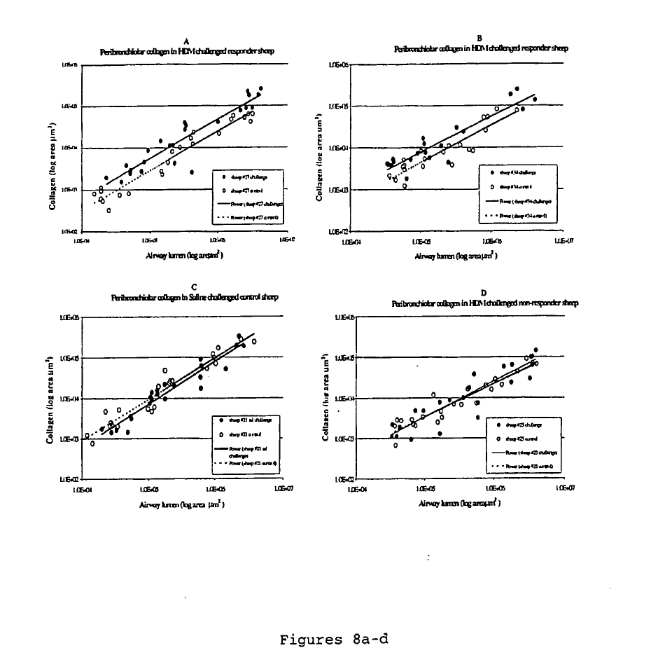

Data from a blinded morphometric analysis show that 50% (2 of 4) of HDM-challenged sheep have statistically significant increases in trichrome stained collagen area assessed by staining with Masson's trichrome in lung compartments chronically challenged with HDM, compared with non-challenged control compartments in the same animal, as shown in FIGS. 8A and B. No such increases were observed in any of the three saline-challenged control sheep or non-responder HDM-challenged sheep, as shown in FIGS. 8C and D. There were also similar significant increases in peribronchiolar connective tissue and smooth muscle, as shown in FIGS. 8E and F, in the challenged lobes of these animals. The increased thickness of airway components in challenged lung compartments was observed in the complete range of bronchiole sizes examined (approx. 200 μm to 2000 μm mean diameter), as shown in FIGS. 8A, B, E and F.

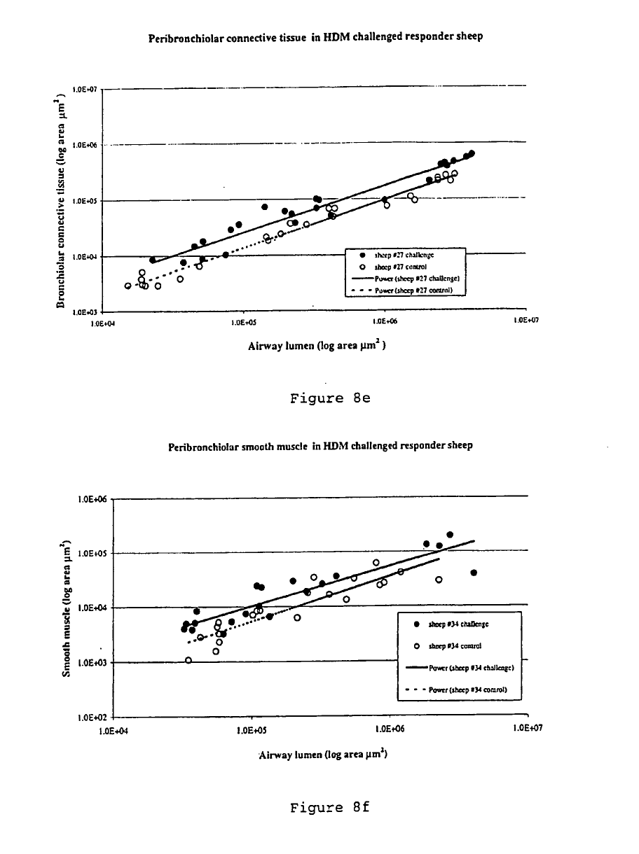

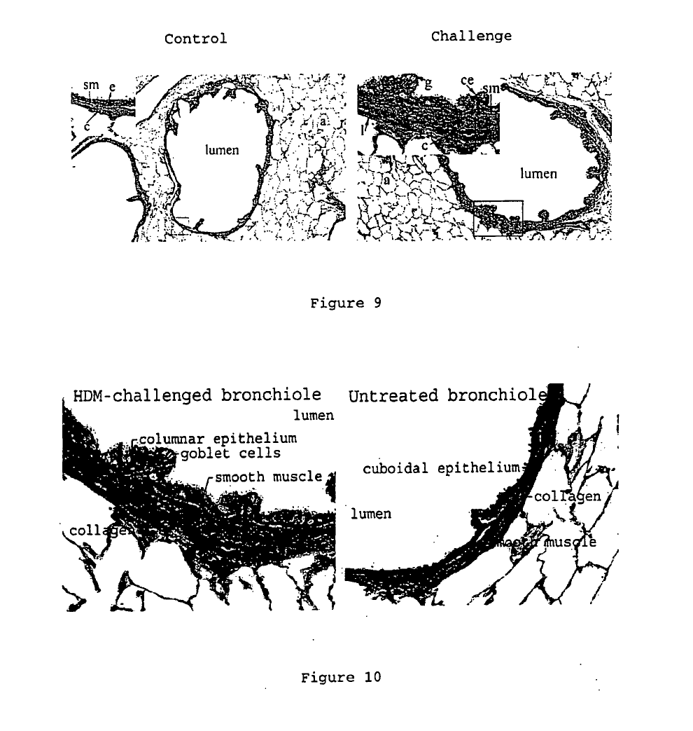

A blinded pathology assessment, performed on coded histological slides of lung tissues taken from these animals, confirmed that there were marked increases in connective tissue in airway walls of bronchioles in the left lung compartments challenged with HDM compared to the connective tissue in equivalent bronchioles of saline challenged control (right) lung compartments. This is illustrated in FIG. 9. In contrast to the control, note in the challenged bronchiole the presence of increased collagen and smooth muscle, increased numbers of goblet cells, and columnar rather than cuboidal epithelium (see high magnification inset of the boxed region).

In the challenged lung, but not in the control lung, there was prominent hyperplasia of alcian blue-stained goblet cells in similar size bronchioles. Bronchiolar epithelial cells lining HDM-challenged bronchioles were columnar, rather than cuboidal as in the bronchioles of the control lung. Lymphocytes were present in the connective tissue surrounding the bronchioles in the challenged lung, but not in controls. This is shown in further detail in FIG. 10. Alcian blue-staining marks the presence of acid mucins. Numerous alcian blue-stained goblet cells (up to 38% of all cells) were observed amongst the cells lining the smaller bronchioles of the challenged lung compartment, while alcian blue-stained goblet cells were absent in similar sized bronchioles of the untreated lung compartment in the same sheep.

These data suggest that there is potential to induce chronic allergic airway changes by administering multiple challenges with HDM to HDM-atopic sheep. The increase in smooth muscle is of particular significance for the validation of the sheep model as it is typically associated with airway remodelling in humans, but is absent in the mouse model (Karol, 1994; Kumar and Foster, 2001).

### EXAMPLE 7

Identification of Proteins Induced by Allergic Sensitisation

The model systems described in Examples 1 and 3 may be used to isolate and identify novel molecules which are specifically expressed by eosinophils. For example, we have found that the expression of a novel galactin, galectin-14, was up-regulated in the lung tissue of sensitized sheep challenged with HDM, and that the protein was released into the BAL fluid.

Screening for cDNA clones which were differentially expressed in fresh and cultured eosinophil-rich mammary lavage (MAL) cells revealed a partial cDNA clone of 325 bp was isolated which showed similarity to the potent human eosinophil chemoattractant ecalectin/galectin-9. Northern blot analysis confirmed that this clone was expressed at relatively high levels by the eosinophil-rich leukocyte population. Therefore an eosinophil-rich MAL cell cDNA library was screened to isolate the full-length clone. Literature and nucleotide data base searches indicate that this molecule is a galactin, but does not show enough identity to known galectins to be classified as the sheep homologue. This galectin can therefore be classified as a novel galectin, and, as it is the fourteenth mammalian galectin published in the data bases, we have designated this molecule galectin-14.

Collection of Mammary Lavage (MAL) Samples

To induce eosinophil migration into the mammary gland, mature non-lactating Merino ewes were primed every 2 weeks by intramammary infusions of 1 mg of solubilized house dust mite extract (HDM, Dermatophagoides pteronyssinus, Commonwealth Serum Laboratories Ltd., Melbourne, Victoria, Australia), rested for 3-4 weeks, and challenged with an intramammary infusion of 1 mg of solubilized HDM. MAL was collected 2 days post-HDM challenge by infusion of sterile pyrogen-free saline (PFS, Baxter Healthcare Pty. Ltd., New South Wales, Australia) followed by milking of the gland, as described in Example 1. Cells were pelleted by centrifugation and washed in PFS. The proportion of eosinophils in the leukocyte suspensions, as determined by Giemsa-stained cytospots, varied from 75 to 90%.

Other sheep received a single intramammary infusion of lipopolysaccharide, and MAL cells were collected at 24 h and 5 days, which resulted in an initial influx of predominantly neutrophils (24 h), followed by macrophage infiltration at day 5.

Collection of Lung Tissue and Bronchoalveolar Lavage (BAL) Samples

4- to 5-month-old parasite-free female merino-cross lambs were sensitized by three subcutaneous injections of 50 μg of HDM, solubilized in PFS with aluminium hydroxide as adjuvant (1:1). Sheep which showed a high HDM-specific IgE serum response were challenged 2-3 weeks later with 1 mg of solubilized HDM, in the lower left lung lobe using a fibre optic bronchoscope (Pentax FG-16x, 5.5 mm OD). The right lung lobe of the same sheep was challenged with PFS only as a control.

BAL samples were collected from each challenge and control lung site before and 6-48 h post-challenge, by gently adding and aspirating 5 ml of PFS through the bronchoscope port. Sheep were sacrificed, and lung tissue samples were collected after the final BAL sample collections (about 48 h post-challenge) for histology. Cells within the BAL were quantified using a Neubauer haemocytometer, and eosinophil numbers were determined on Giemsa-stained cytospots.

Larger BAL leukocyte populations required for RNA preparation were collected from whole lung lavage of left and right lung lobes by occluding the entrance to one lung lobe with a Foley catheter as described previously (Dunphy et al., 2001). Lung tissue was also collected from each lung lobe for RNA preparation and histology.

Peripheral Blood Leukocytes

Peripheral blood was drawn from the jugular vein of sheep into plastic tubes containing EDTA-Na₂ (BDH Merck, Victoria, Australia). Red blood cells were lysed with TAC (0.17 M Tris/0.16 M NH4Cl, pH 7.2) at 37° C., and the remaining leukocytes were washed in PBS, and resuspended in 1% BSA/PBS.

RNA Preparation

Total RNA was purified from 0.1-1 g of tissue or approximately 1×10⁸ cells, using a standard guanidinium thiocyanate, phenol/chloroform extraction (Chomczynski and Sacchi, 1987).

Low Stringency RT-PCR

cDNA clones differentially expressed by fresh and cultured cells were amplified from eosinophil-rich MAL cells by low stringency RT-PCR, using the displayPROFILE kit from Display Systems Biotech (Integrated Sciences, Sydney, Australia) as described in the kit manual (version 2.0). Total RNA from eosinophil-rich MAL cells of nematode challenged sheep (Dunphy et al., 2001) was used as template. The PCR primer which resulted in amplification of the partial galectin-14 cDNA was DisplayPROBEsEu4, whose sequence is set out in Table I. PCR products of interest were re-amplified and subcloned into pGEM-Teasy (Promega) before being sequenced using the BIG DYE terminator mix (PerkinElmer Life Sciences).

TABLE I

Primers and adapters utilized in low stringency and
conventional RT-PCR

| | | Annealing |
|---|---|---|
| Name | Sequence | temperature (° C.) |
| O-extension primer | GGTACCGCAGTCTACGAGACCAGT | 55-60 |
| DisplayPROBEsEu4 | ATGAGTCCTGACCGAAAG | 55-60 |
| G14 5′UTR | ATTCCTGTTGCAGAAGTCTACCTGGACA | 54 |
| G14 3′UTR | GAACATCTTCCACACGGTAGGGGT | 54 |
| G14 5′pGEX | AGGATCCATGCAGAGCGAAAGTGGTCACGA | 59 |
| G14 3′pGEX | CGGCGGCCGCTTAAATCTGGAAGCTGATAT | 59 |

The sequences of adapters and primers used in low stringency or conventional RT-PCR are shown. Annealing temperatures utilized in PCRs are also indicated. The G14 3′UTR primer and G14 3′pGEX primer were used as both RT and downstream PCR primers. Nucleotide substitutions introduced in primers G14 5′pGEX and G14 3′pGEX to alter codon usage to that preferred by E. coli are shown in bold.

Construction and Screening of a MAL cDNA Library

The SMART cDNA library construction kit (CLONTECH) was used as recommended by the manufacturer to prepare a cDNA library representing mRNA expressed in an eosinophil-rich leukocyte population, as described previously (Dunphy et al., 2001). LE392 cells were infected with the pTriplEx2 phage library, and the plaques screened with the original galectin-14 partial RT-PCRcDNA. The 32P-labeled galectin-14 cDNA probes were generated from the RT-PCR clone using Klenow polymerase and the Giga-prime kit (Bresatech, Adelaide, Australia). The hybridization and wash conditions used were the same as for Northern blot analysis (see below). At least 1×10⁶ plaque-forming units were used for each primary screen. Once individual plaques of interest were isolated in tertiary screens, the λTriplEx2 phage was converted into pTriplEx2 plasmid, as instructed in the SMART cDNA manual. The cDNAs were then sequenced using the 5′-sequencing primer of pTriplEx2 (Invitrogen).

Amplification of Galectin-14 cDNA Containing the Full Putative Coding Region

Two RT-PCR primers were designed within the putative 5′- and 3′-untranslated regions (UTRs) of galectin-14 (G145′UTR and G143′UTR; see Table I). Approximately 1.25 μg of MAL cell total RNA was used as a template for reverse transcriptase. 2-10 μl of the RT mix was used as a template for 30 PCR cycles. The PCR used 0.25 μM of each primer, 200 μM of each dNTP, and 2.5 units of Taq polymerase in a total volume of 100 μl. The 30 PCR cycles utilized a denaturation step of 95° C. for 30 s, an annealing temperature of 54° C. for 1 min, and an extension temperature of 74° C. for 1 min. An additional denaturation of 5 min preceded the 30 cycles, and a prolonged extension of 10 min completed the PCR. PCR products were subcloned into pGEM-Teasy and sequenced as described above.

Northern Blot Hybridizations

Approximately 10 μg of total RNA was transferred to Hybond N+ membranes (Amersham Biosciences, Inc.) by capillary action. Membranes were prehybridized for 4 h at 42° C. in Church buffer (0.5 M sodium phosphate, pH 7.2/1% BSA/7% SDS/2 mM EDTA). ³²P-Labeled galectin-14 cDNA probes were generated as described above. Probes were hybridized to the membranes in Church buffer overnight at 65° C. The membranes were washed at high stringency in 0.2×SSC/0.1% SDS at 37-42° C.

Production of Recombinant Galectin-14 in Escherichia coli

To produce recombinant galectin-14, the entire coding region of the mRNA was amplified by RT-PCR, and subcloned into the E. coli GST expression vector pGEX-6P-2 (Amersham Biosciences, Inc.). The RT-PCR primers used incorporated four nucleotide changes to alter codon usage to that preferred by E. coli (Kane, 1995), as shown in Table I. The protease-deficient E. coli strain BL-21 was used to express recombinant galectin-14 in the form of a GST fusion protein. Expression was induced by addition of 0.1 mM isopropyl-1-thio-o-D-galactopyranoside for 2-3 h at 34° C. The fusion protein was then isolated using a glutathione-Sepharose column and cleaved with PreScission protease (Amersham Biosciences, Inc.) on the column as instructed by the manufacturer.

The cleaved and purified galectin-14 recombinant protein contained an additional 5 amino acids at its amino terminus (remnants of the vectors cleavage and multiple cloning sites; GPLGS). The relative purity of the protein preparation was confirmed by Coomassie Blue-stained reducing SDS-PAGE, as shown in FIG. 12, and amino terminal sequence of the protein was determined in order to confirm that it was in-frame and cleaved appropriately. The amino-terminal sequencing confirmed the first 24 residues, and indicated that the preparation was relatively pure. The complete sequence of galectin-14 is disclosed in Australian provisional patent application No. PR6747 by The University of Melbourne, filed on 23 Jul. 2001, and in Dunphy et al., 2002.

Eosinophil-specific Expression of Galectin-14

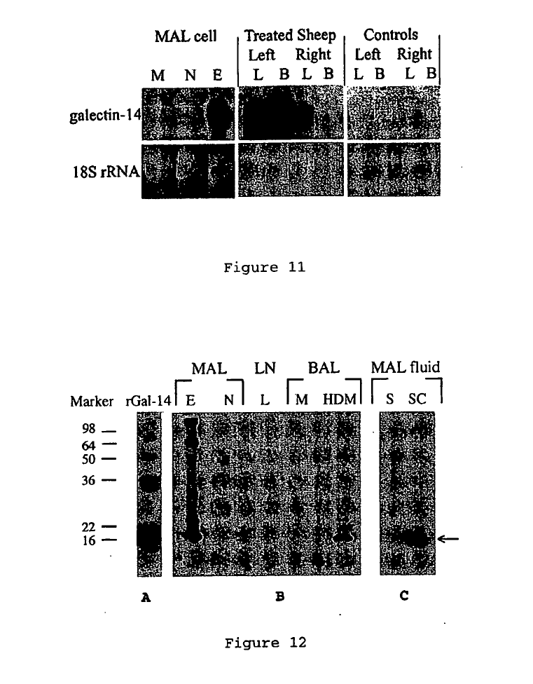

Northern blot analysis detected relatively high levels of galectin-14 mRNA in eosinophil-rich leukocyte populations recovered from the mammary lavage after intramammary infusions of HDM, as shown in FIG. 11. Galectin-14 mRNA was not detected in macrophage- or neutrophil-rich MAL leukocyte populations induced by lipopolysaccharide intramammary infusions, indicating that the gene may be specifically expressed by eosinophils and not by other leukocyte populations. To study the expression of the galectin-14 protein, monoclonal antibodies (mabs) were raised against cleaved and purified recombinant galectin-14. BALB/c mice were given intraperitoneal injections of about 5 μg of cleaved and purified recombinant galectin-14 once a month for 3 months, initially in complete Freund's adjuvant and subsequently in incomplete Freund's adjuvant. Spleen cells from immune mice were fused with NS-1 myeloma cells using 50% polyethylene glycol 4000 (Merck, Darmstadt, Germany), and supernatants screened for galectin-14 binding by enzyme-linked immunosorbent assay. Positive hybridomas were cloned by limiting dilution at least three times before being converted to DM10 media alone. Ascitic fluid was produced by giving pristine-primed BALB/c mice an intraperitoneal injection of 1×10⁷ hybridoma cells. A mAb with high reactivity for galectin-14 but no cross-reactivity with another ovine galactin, OVGAL11, (Dunphy et al., 2000)) was selected and used to study endogenous galectin-14 protein expression.

Eosinophil-rich MAL and BAL cells solubilized in sample buffer were run on SDS-PAGE, transferred to nitrocellulose, and probed with the galectin-14 mAb. This clearly detected a protein of similar size to recombinant galectin-14 under both reducing and non-reducing conditions (apparent molecular mass about 17 kDa), as shown in FIG. 12. The expected molecular mass of galectin-14, calculated from the predicted amino acid sequence, is only slightly larger (18.2 kDa). In concentrated samples or after storage, higher molecular weight bands could often be observed in both recombinant and endogenous samples, probably due to aggregation. These aggregates did not dissociate, even when samples were run on gels under reducing conditions. Occasionally, higher molecular weight bands were detected by galectin-14 mAb which did not correspond to the predicted mass of oligomers, especially in samples which contained relatively large amounts of monomeric galectin-14. These may be the result of post-translational processing of galectin-14 or due to galectin-14 forming stable complexes with other cellular proteins. In agreement with the Northern blot analysis, Western blots did not, or only weakly, detect galectin-14 in neutrophil-or macrophage-rich cell populations, or in lymph node lymphocytes. The weak reactions observed in some neutrophil and lymphocyte preparations were probably due to contaminating (1-2%) eosinophils present in these populations being detected by the highly sensitive enzyme chemiluminescence assay, because no staining was observed in these cells on cytospots.

Detailed examination of cytospots prepared from circulating blood cells and eosinophil-rich MAL or BAL cells of HDM-sensitized and challenged sheep confirmed the localization of galectin-14 to eosinophils and not neutrophils or lymphocytes. The galectin-14 staining in eosinophils was patchy and widespread within the cytoplasm, with occasional staining of the nuclei, but did not appear to localize to the granules. Flow-cytometry analysis detected strong galectin-14 intracellular staining in more than 95% of eosinophils isolated from mammary lavage after allergen challenge. In contrast, no intracellular staining was detected in neutrophils and macrophages, and only weak nonspecific staining in lymphocytes. The nonspecific nature of the absorbance shift in lymphocytes was confirmed by negative staining of lymphocytes in both cytospots and lymph node sections. No galectin-14 surface staining was detected on eosinophils or any other class of leukocytes.

Relatively high levels of galectin-14 mRNA were detected in lung tissue and BAL cells of HDM sensitised and lung-challenged sheep, as shown in FIG. 11. There were consistently higher levels of galectin-14 mRNA in the lung tissue and BAL of the left, challenged lung lobe, compared with the samples from the right control lobe. The level of expression was associated with lung eosinophilia, with the sheep known to have the greatest number of BAL eosinophils (38%) exhibiting the highest levels of galectin-14 mRNA. Weak or no expression was observed in the lungs of control, unchallenged sheep. Galectin-14 protein was also detected by Western blot analysis, in the cell-free BAL fluid of HDM-challenged lung compartments.

Galectins are carbohydrate-binding proteins which have been increasingly implicated in both adaptive and innate immune responses. The eosinophil-specific expression of galectin-14 and its secretion into the lumen of the lung in the sheep asthma model indicates that it may play an important role in regulating the activity of eosinophils during allergic responses, and further highlights the importance of carbohydrate binding proteins during inflammation and the use of the sheep model to examine expression of novel target molecules.

### EXAMPLE 8

Identification of Differentially Expressed Proteins by Representational Difference Analysis (RDA)

Representational difference analysis (RDA) is performed as described previously [Dunphy et al, 2000]. RNA prepared from control mammary gland or lung tissue is used as the driver. RNA prepared from the corresponding tissue of a sheep sensitised with HDM as described in Example 1 or Example 3 respectively, collected 2 days post-challenge, is used as the tester. Double stranded cDNA is produced using the Superscript Choice System (GIBCO BRL Life Technologies, Melbourne Australia). The double stranded cDNA is digested with Sau3A and ligated to annealed adaptors and amplified by PCR. Three rounds of subtractive hybridization PCRs are completed before individual PCR bands are subcloned into the pGEM-Teasy vector (Promega, Sydney, Australia) and sequenced using the Big Dye sequencing kit (Perkin Elmer Applied Biosystems, Melbourne, Australia).

It will be appreciated that microarray or proteomic, or glycomic methods, which can differentiate between different tissue phenotypes, may also be used. Suitable methods will be well known to those skilled in the art. See for example Zou et al. (2002).

### EXAMPLE 9

Changes in Airway Flow Resistance in Sheep Challenged with Aerosolised HDM

Physiological asthmatic responses in animals administered inhalation challenges of HDM were assessed by measuring changes in resistance to pulmonary airflow.

Three sheep, previously sensitised to HDM, as described in example 3, were challenged in the lungs by inhalation of aerosolized HDM, by nebulizing 3 ml of 2.5 mg/ml HDM into an inhalation apparatus. The inhalation apparatus consisted of a nebulizer connected to a T-piece plastic tube of 1 cm diameter, which joined an endotracheal tube inserted via the nasal cavity into the trachea, with a 2 litre rebreathing bag filled with oxygen. The sheep were allowed to voluntarily inhale the HDM/oxygen mixture for a period of 2 minutes (approximately 30 natural breaths).

Preliminary lung mechanics data was gathered from conscious and unsedated sheep, which were appropriately restrained in a custom made body sheath and head restraining harness tethered in a metabolism cage. Physiological data was collected from specialised tracheal and oesophageal balloon catheters, which measure intra- and extra-airway pressures respectively. The oesophageal and tracheal catheters were connected to a differential gas transducer to measure transpulmonary pressure. Flow measurements were obtained via a pneumotachograph attached to the proximal end of the endotracheal tube. Mean pulmonary resistance to airflow was assessed by dividing airflow by the transpulmonary pressure. Airways resistance was measured both before the HDM inhalation challenge (baseline data), and at sequential times up to 20 minutes after the inhalation HDM challenge.

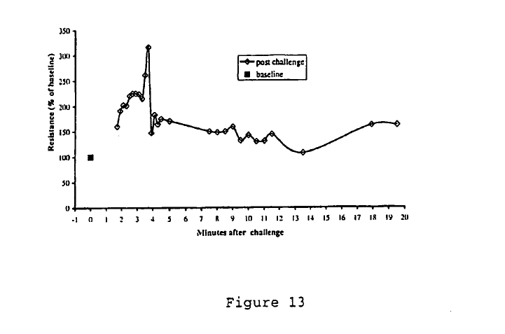

Pulmonary airflow resistance values increased after inhalation HDM challenges in all three sheep tested. An inhalation challenge with 3 mls of saline did not significantly increase airways resistance over a twenty minute period, indicating that the response is specific to HDM (data not shown) In the example shown in FIG. 13, increased airflow resistance peaked sharply about four minutes after the HDM inhalation challenge (approximately 350% change). The resistance values then gradually declined towards the prechallenge baseline value. The results indicate that HDM can induce asthmatic physiological responses, such as bronchoconstriction in sheep, similar to those observed in human asthma.

It will be evident to the person skilled in the art that novel processes or molecules discovered using the experimental systems of the invention will be useful as potential targets for the development of new drugs and therapeutic strategies for the treatment of asthma.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### References

Abraham W M, Delehunt J C, Yerger L, Marchette B. (1983). Characterization of a late phase pulmonary response after antigen challenge in allergic sheep. Am Rev Respir Dis, 128: 839-844.
Abraham W M, Gill A, Ahmed A, Sielczak M W, Lauredo I T, Botinnikova Y, Lin K C, Pepinsky B, Leone D R, Lobb R R, Adams S P. (2000). A small-molecule, tight-binding inhibitor of the integrin alpha(4)beta(1) blocks antigen-induced airway responses and inflammation in experimental asthma in sheep. Am J Respir Crit Care Med, 162: 603-611.
Balic A, Bowles V M, Meeusen E N. (2000). Cellular profiles in the abomasal mucosa and lymph node during primary infection with Haemonchus contortus in sheep. Vet Immunol Immunopathol, 75: 109-120.
Bice D E, Seagrave J, Green F H. (2000). Animal models of asthma: potential usefulness for studying health effects of inhaled particles. Inhal Toxicol, 12: 829-862.
Bischof R J and Meeusen E N T (2002). Cellular kinetics of an allergic-type response in a sheep mammary gland model of inflammation. Clin. Exp. Allergy 32: 619-626.
Butterworth A E, Thorne J I. (1993). Eosinophils and Parasitic Diseases, in Immunopharmacology of Eosinophils, H. Smith and R. M. Cook, Editors. (Academic Press) p. 119-192.
Chomczynski P. and Sacchi N. 1987. Single-step method of RNA isolation by acid guanidinium thiocyanate-phenol-chloroform extraction. Anal. Biochem. 162: 156-159.
Desreumaux P, Capron M. (1996). Eosinophils in allergic reactions. Curr Opin Immunol, 8: 790-5.
Duffus W P H, Franks D. (1980). Killing of juvenile Fasciola hepatica by purified bovine eosinophil proteins. Clinical Experimental Immunology, 40: 430-440.
Dunphy J L, Balic A, Barcham G J, Horvath A J, Nash A D, Meeusen E N (2000) Isolation and characterization of a novel inducible mammalian galectin. J Biol Chem. 275(41):32106-13.
Dunphy J L, Barcham G J, Bischof R J, Young A R, Nash A Meeusen E N (2002) Isolation and characterization of a novel eosinophil-specific galectin released into the lungs in response to allergen challenge. J Biol Chem. 277(17):14916-14924.
Dunphy J, Horvath A, Barcham G, Balic A, Bischof R and Meeusen E (2001). Isolation, characterisation and expression of mRNAs encoding the ovine CC chemokines, monocyte chemoattractant protein (MCP)-1 alpha and -2. Vet. Immunol. Immunopathol., 82: 153-164.
Fath M A, Wu X, Hileman R E, Linhardt R J, Kashem M A, Nelson R M, Wright C D, Abraham W M. (1998). Interaction of secretory leukocyte protease inhibitor with heparin inhibits proteases involved in asthma. J Biol Chem, 273: 13563-13569.
Foster P S, Mould A W, Yang M, Mackenzie J, Mattes J, Hogan S P, Mahalingam S, Mckenzie A N, Rothenberg M E, Young I G, Matthaei K I, Webb D C (2001). Elemental signals regulating eosinophil accumulation in the lung. Immunol Rev 2001 Feb;179:173-81
Fujimoto K, Kubo K, Okada K, Kobayashi T, Sekiguchi M, Sakai A. (1996). Effect of the 21-aminosteroid U-74006F on antigen-induced bronchoconstriction and bronchoalveolar eosinophilia in allergic sheep. Eur Respir J, 9: 2044-2049.
Greenhalgh C J, Jacobs H J, Meeusen E N T. (1996). An in vivo mammary infusion model for tissue migration of leucocytes during inflammation. Immunol Cell Biol, 74: 497-503.
Grunig G, Warnock M, Wakil A E, Venkayya R, Brombacher F, Rennick D M, Sheppard D, Mohrs M, Donaldson D D, Locksley R M, Corry D B. (1998). Requirement for IL-13 independently of IL-4 in experimental asthma. Science, 282: 2261-2263.
Jones D G. (1993). The eosinophil. J Comp Pathol, 108: 317-35.
Karol M H. (1994). Animal models of occupational asthma. Eur Respir J, 7: 555-568.
Kumar R K and Foster P S. (2001).Immunol Cell Biol, 79: 141-144.
Lee C S, Gogolin-Ewens K, White T R, Brandon M R. (1985). Studies on the distribution of binucleate cells in the placenta of the sheep with a monoclonal antibody SBU-3. J Anat, 140: 565-576.
Menegazzi R, Zabucchi G. et al. (1992). “A new, one-step assay on whole cell suspensions for peroxidase secretion by human neutrophils and eosinophils.” J Leukoc Biol, 52: 619-24.
Naessens J, Howard C, Hopkins J. (1997). Nomenclature and characterization of leukocyte differentiation antigens in ruminants. Immunology Today 18:365-368.
Pastoret P-P, Griebel P, Bazin H, Govaerts A. (eds) (1998). Chapter XIV, “Sheep Immunology and Goat Peculiarities” in “Handbook of Vertebrate Immunology” (Academic Press).
Rainbird M A, Macmillan D, Meeusen E N. (1998). Eosinophil-mediated killing of Haemonchus contortus larvae: effect of eosinophil activation and role of antibody, complement and interleukin-5. Parasite Immunol. 20: 93-103.
Schelegle E S et al. (2001). Allergic Asthma Induced in Rhesus Monkeys by House Dust Mite (Dermatophagoides farinae). American J Path. 158: 333-341.
Villinger F. et al. (1995). Comparative sequence analysis of cytokine genes from human and non-human primates. J. Immunol. 155: 3946-3954.
Walker C, Kaegi M K Braun P. Blaser K. (1991). Activated T cells and eosinophilia in bronchoalveolar lavages from subjects with asthma correlated with disease severity. J Allergy Clin Immunol, 88: 935-42.
Wills-Karp M, Luyimbazi J, Xu X, Schofield B, Neben T Y, Karp C L, Donaldson D D. (1998). Interleukin-13: central mediator of allergic asthma. Science, 282: 2258-2261.
Zou J, Young S, Zhu F, Xia L, Skeans s, Wan Y, Wang L, McClanahan T, Gheyas F, Wei D, Garlisi C, Jakway J, Umland S. (2002) Identification of Differentially Expressed Genes in a Monkey Model of Allergic Asthma by Microarray Technology. Chest, 121:26S-27S.

1-32. (canceled).

33. An in vivo model system for an allergic condition, comprising a mammal which has been subjected to sensitisation with an antigen or administration of a cytokine involved in response to allergen, in which
a) the mammal is a female, and is sensitised by repeated administration of the antigen into the mammary gland; or
b) the mammal is of either sex, and is sensitised by administration of the antigen, followed by administration directly to the lung; or
c) the mammal is of either sex, and blood and tissue eosinophilia is induced by administration of a cytokine involved in response to allergen,
in which the mammal is a member of the order Artiodactyla, and the antigen is not one derived from a helminth parasite.

34. A model according to claim 33, in which the mammal is of either sex, and is sensitised by administration of the antigen, followed by administration directly to the lung, and the allergic condition is one which is associated with eosinophilia and elevated levels of IgE.

35. A model according to claim 33, in which the antigen is selected from the group consisting of house dust mite, animal dander, feathers, plant antigens, moulds, and household or industrial chemicals.

36. A model according to claim 35, in which the antigen is house dust mite.

37. A model according to claim 35, in which the antigen is an extract of house dust mite.

38. A model according to claim 35, in which the animal dander is selected from the group consisting of cat dander, dog dander, bird dander and cockroach dander.

39. A model according to claim 35, in which the plant antigens are selected from the group consisting of grass pollens or tree pollens.

40. A model according to claim 39, in which the grass pollens are ryegrass pollen or Alternaria pollen.

41. A model according to claim 39, in which the tree pollens are birch or cedar pollens.

42. A model according to claim 33, in which the antigen is associated with asthma in humans.

43. A model according to claim 33, in which the cytokine involved in response to allergen is interleukin-5.

44. A model according to claim 33, in which the cytokine involved in response to allergen is eotaxin.

45. A lung model according to claim 33, in which the antigen or molecule involved in response to allergen is administered by intravenous, oral, subcutaneous, intradermal or intramuscular administration, followed by administration directly into the lung.

46. A model according to claim 45, in which the mammal is a ruminant or a pig.

47. A model according to claim 46, in which the mammal is a sheep, goat, or bovine.

48. A model according to claim 46, in which the mammal is a sheep or a goat.

49. A method of preparing a model according to claim 33, comprising the step of administration of antigen or of a cytokine involved in response to allergen to a mammal, thereby to induce a specific allergic response characterised by the recruitment of inflammatory cells into the blood of the mammal.

50. A method according to claim 49, comprising the step of repeated administration of antigen into the mammary gland of a mammal, thereby to induce a specific allergic response characterised by the recruitment of inflammatory cells into the mammary gland of the mammal.

51. A method according to claim 49, comprising the step of repeated administration of antigen into the lung of a mammal, thereby to induce a specific allergic response characterised by the recruitment of inflammatory cells into the lung of the mammal.

52. A method according to claim 49, further comprising the step of collection of the inflammatory cells.

53. A method according to claim 49, in which the administration is intravenous, oral, subcutaneous, intradermal, or intramuscular.

54. A method according to claim 50, in which the administration is subcutaneous.

55. A method according to claim 51, in which the administration to the lung is via a fibre-optic bronchoscope or nebulizer.

56. A method according to claim 51, in which the animal is a ruminant or a pig.

57. A method according to claim 56, in which the mammal is a sheep, goat, or bovine.

58. A method according to claim 57, in which the mammal is a sheep or a goat.

59. Use of a model according to claim 33 for:
a) the study of asthma;
b) the examination of the effects of chronic allergen exposure;
c) in vivo testing of the efficacy of candidate drugs for the treatment of asthma;
d) in vivo screening or testing of new anti-inflammatory drugs, therapies, and/or procedures; or
e) in vitro screening assays for the development of new anti-inflammatory or anti-eosinophil degranulation drugs.

60. Use according to claim 59, in which candidate targets for anti-allergic drug targets are identified using molecular or biochemical techniques.

61. Use according to claim 60, in which the techniques are genomic, proteomic, or glycomic techniques.

62. Use according to claim 60, in which the techniques are differential display, representational difference analysis, microarrays, or 2-dimensional electrophoresis.

63. Inflammatory cells obtained by a method according to claim 52.

64. Use of inflammatory blood or MAL cells according to claim 63 for:
a) the identification of processes or molecules differentially active or expressed in “activated” and “non-activated” eosinophils and/or other inflammatory cells;
b) identification of processes and molecules involved in the recruitment of eosinophils and/or other inflammatory cells;
c) identification of processes and molecules involved in degranulation of eosinophils and/or other inflammatory cells;
d) in vivo testing of the efficacy of candidate drugs for the treatment of asthma;
e) in vivo screening and testing of new anti-inflammatory drugs, therapies, and/or procedures; or
f) in vitro screening assays for the development of new anti-inflammatory or anti-eosinophil degranulation drugs.
